SEQUENCE LISTING (1270 sequences)

SEQ ID NO: 1 - DNA, 621 bp, synthetic construct
  misc_feature 1..621: note = artificial sequence
  source 1..621: mol_type = other DNA
atggccgtttacccatacgatgttcctgactatgcgggctatccctatgacgtcccggactatgcaggctcctatccatatgacgttccagattacgctggatctggcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 2-1267 - skipped (1266 entries)

SEQ ID NO: 1268 - DNA, 22 bp, synthetic construct
  misc_feature 1..22: note = Profiling 1
  source 1..22: mol_type = other DNA
ttctagagcggccgcttcgagc

SEQ ID NO: 1269 - DNA, 22 bp, synthetic construct
  misc_feature 1..22: note = Profiling 2
  source 1..22: mol_type = other DNA
cgctgatctcacgccgtggtga

SEQ ID NO: 1270 - DNA, 22 bp, synthetic construct
  misc_feature 1..22: note = Tailing
  source 1..22: mol_type = other DNA
agttccgcgtacgtacggcgtc

(19) United States
(12) Patent Application Publication
(10) Pub. No.: US 2026/0201365 A1
(43) Pub. Date: Jul. 16, 2026

(54) Systems and Methods to Assess RNA Stability

(71) Applicant: The Board of Trustees of the Leland Stanford Junior University, Stanford, CA (US)

(72) Inventors: Maria Barna, Emerald Hills, CA (US); Rhiju Das, Palo Alto, CA (US); Kathrin Leppek, Bonn (DE); Gun Woo Byeon, Stanford, CA (US); Eesha Sharma, Stanford, CA (US)

(73) Assignee: The Board of Trustees of the Leland Stanford Junior University, Stanford, CA (US)

(21) Appl. No.: 19/306,922

(22) Filed: Aug. 21, 2025

Related U.S. Application Data
(63) Continuation of application No. 17/456,353, filed on Nov. 23, 2021, now abandoned.
Application No. 17/456,353 is a continuation of application No. PCT/US2021/040027, filed on Jul. 1, 2021.
(60) Provisional application No. 63/051,269, filed on Jul. 13, 2020.
(60) Provisional application No. 63/072,669, filed on Aug. 31, 2020.
(60) Provisional application No. 63/165,662, filed on Mar. 24, 2021.

(51) Int. Cl. C12N 15/10; C12Q 1/6806; G01N 27/447

(52) U.S. Cl. CPC C12N 15/1065; C12N 15/1089; C12N 15/1096; C12Q 1/6806; G01N 27/44791

(57) ABSTRACT

Systems and methods for assessing mRNA in vivo and/or in vitro stability are disclosed. Some embodiments methods obtain RNA indexed or barcoded RNA molecules which are then tested against various conditions including stability inside of cells, stability in cell lysate, and stability in solution (e.g., for storage and/or transportation). Additional embodiments describe methods to determine degradation points with single base resolution.

### CROSS-REFERENCE TO RELATED APPLICATIONS

The current application is a continuation of U.S. patent application Ser. No. 17/456,353, filed Nov. 23, 2021, which claims priority to U.S. Provisional Patent Application No. 63/051,269, filed Jul. 13, 2020, U.S. Provisional Patent Application No. 63/165,662, filed Mar. 24, 2021, U.S. Provisional Patent Application No. 63/072,669, filed Aug. 31, 2020, and International Application PCT/US2021/040027, filed Jul. 1, 2021, the disclosures of which are hereby incorporated by reference in their entireties.

### FIELD OF THE DISCLOSURE

The present invention relates to ribonucleic acid (RNA). More specifically, the present invention relates to RNA molecules with enhanced stability and translation and assessment thereof, and further relates to systems and methods to enhance RNA stability and translation.

### INCORPORATION OF SEQUENCE LISTING

This application hereby incorporates by reference the material of the electronic Sequence Listing filed concurrently herewith. The material in the electronic Sequence Listing is submitted as an XML (.xml) file entitled “06754PCT_Seq_List_ST26.xml” created on Aug. 21, 2025, which has a file size of 258,422 bytes, and is herein incorporated by reference in its entirety. No new matter is entered by this Sequence Listing.

### BACKGROUND OF THE DISCLOSURE

There are multiple problems with prior methodologies of effecting protein expression. For example, introduced DNA can integrate into host cell genomic DNA at some frequency, resulting in alterations and/or damage to the host cell genomic DNA. Alternatively, the heterologous deoxyribonucleic acid (DNA) introduced into a cell can be inherited by daughter cells (whether or not the heterologous DNA has integrated into the chromosome) or by offspring.

In addition, assuming proper delivery and no damage or integration into the host genome, there are multiple steps which must occur before the encoded protein is made. Once inside the cell, DNA must be transported into the nucleus where it is transcribed into RNA. The RNA transcribed from DNA must then enter the cytoplasm where it is translated into protein. Not only do the multiple processing steps from administered DNA to protein create lag times before the generation of the functional protein, each step represents an opportunity for error and damage to the cell. Further, it is known to be difficult to obtain DNA expression in cells as DNA frequently enters a cell but is not expressed or not expressed at reasonable rates or concentrations. This can be a particular problem when DNA is introduced into primary cells or modified cell lines.

Attempts have been made to use RNA and messenger RNA (mRNA) as therapeutic agents. However, RNA is generally unstable and highly susceptible to degradation.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

In one embodiment, a method to determine RNA stability includes obtaining a pool of RNA molecules, where each RNA molecule is uniquely encoded with a barcoding sequence and each barcoding sequence is flanked by at least one profiling sequence, treating the pool of RNA molecules under an experimental condition, and isolating the pool of RNA molecules at a specified timepoint to generate a fraction of RNA molecules showing stability under the experimental condition for the specified timepoint.

In a further embodiment, the method further includes sequencing the barcode sequence of each RNA molecule in the fraction to identify the presence of each RNA molecule in the fraction of RNA molecules.

In another embodiment, the method further includes stability of the RNA molecules associated with each barcode sequence in the fraction by identifying the prevalence of each barcode in the fraction.

In a still further embodiment, the treating step includes transfecting the pool of RNA molecules into a collection of cells.

In still another embodiment, the cells are selected from mammalian cells, yeast cells, bacteria cells, and plant cells.

In a yet further embodiment, the treating step includes adding the pool of RNA molecules to a cell lysate.

In yet another embodiment, the treatment condition is selected from temperature, pH, presence of certain molecules, presence of certain ions, concentration of certain molecules, concentration of certain ions, irradiation, buffer type, and buffer concentration.

In a further embodiment again, the method further includes size selecting for full-length RNA molecules.

In another embodiment again, size selecting includes one or more of agarose gel electrophoresis, polyacrylamide gel electrophoresis, and capillary electrophoresis.

In a further additional embodiment, size selecting includes treating the RNA molecules with a 5′-3′ nuclease that is inhibited by the presence of a 5′ cap moiety.

In a still further additional embodiment, size selecting includes performing reverse transcription PCR to amplify full-length RNA molecules.

In another additional embodiment, the isolating step further includes isolating the pool of RNA molecules at a second specified timepoint to generate a second fraction of RNA molecules showing stability under the experimental condition for the specified timepoint.

In a still yet further embodiment, a method to identify a degradation site within an RNA molecule includes obtaining a pool of RNA molecules, wherein the RNA molecules encode for a sequence of interest, treating the pool of RNA molecules under an experimental condition to degrade the RNA molecules in the pool of RNA molecules, isolating the pool of RNA molecules at a specified timepoint, ligating an adapter to one end of the degraded RNA molecules in the pool of RNA molecules, and sequencing the ligated and degraded RNA molecules in the pool of RNA molecules to identify the degradation locations in the pool of RNA molecules.

In still yet another embodiment, the adapter is ligated to the 5′ end of the degraded RNA molecules.

In a still further embodiment again, the treatment condition is selected from temperature, pH, presence of certain molecules, presence of certain ions, concentration of certain molecules, concentration of certain ions, irradiation, buffer type, and buffer concentration.

In still another embodiment again, the pool of RNA molecules includes a plurality of sequences of interest, wherein each sequence of interest is uniquely encoded with a barcoding sequence and each barcoding sequence is flanked by at least one profiling sequence.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates exemplary results of in vitro versus in vivo RNA stability in accordance with various embodiments.

FIG. 2 illustrates a generalized structure of RNA molecules in accordance with various embodiments of the invention.

FIG. 3 illustrates a method to screen RNAs for increased in vivo stability in accordance with various embodiments of the invention.

FIG. 4A illustrates a method to screen RNAs for increased in vitro stability in accordance with various embodiments of the invention.

FIG. 4B illustrates an exemplary degradation curve in accordance with various embodiments of the invention.

FIG. 5 illustrates a method to identify single nucleotide resolution of degradation in accordance with various embodiments of the invention.

FIG. 6 illustrates a method to identify RNAs possessing increased in vivo and/or in vitro stability in accordance with various embodiments of the invention.

FIG. 7 illustrates exemplary data analyzing full length RNAs present after 22 hours of in vitro testing in accordance with various embodiments of the invention.

FIGS. 8A-8D illustrates exemplary data of a heatmap showing RNA dropout after 22 hours of in vitro testing in accordance with various embodiments of the invention. Specifically, FIG. 8A illustrates a full view of the heatmap and column and row labels; while FIG. 8B illustrates an enlarged view of the heatmap; FIG. 8C illustrates the row labels; and FIG. 8D illustrates the column labels.

### DETAILED DESCRIPTION OF THE DISCLOSURE

Turning now to the drawings, systems and methods to quantify RNA stability and uses thereof are provided. Many embodiments provide RNA molecules, including messenger RNA (mRNA), that allow for an assessment of in vitro and/or in vivo stability. Further embodiments provide methods and systems to assess such stability as well as provide single base resolution of degradation products.

In vivo and in vitro stability are two independent problems for RNA. In vivo stability can depend on untranslated sequences at 3′-ends of mRNAs, structures and sequences that signal decay, process that identify premature stop codons, RNA elements recognized by cellular endonucleases and exonucleases, and ribosome-dependent decay processes. (See, e.g., Koh, W. S., Porter, J. R. & Batchelor, E. Tuning of mRNA stability through altering 3′-UTR sequences generates distinct output expression in a synthetic circuit driven by p53 oscillations. Sci Rep 9, 5976 (2019). doi: 10.1038/s41598-019-42509-y; Park E, Maquat L E. Staufen-mediated mRNA decay. Wiley Interdiscip Rev RNA. 2013 July-August; 4(4):423-35. doi: 10.1002/wrna.1168. Epub 2013 May 16. PMID: 23681777; PMCID: PMC3711692; Brogna, S., Wen, J. Nonsense-mediated mRNA decay (NMD) mechanisms. Nat Struct Mol Biol 16, 107-113 (2009). doi: 10.1038/nsmb.1550; Blandine C. Mercier, Emmanuel Labaronne, David Cluet, Alicia Bicknell, Antoine Corbin, Laura Guiguettaz, Fabien Aube, Laurent Modolo, Didier Auboeuf, Melissa J. Moore, Emiliano P. Ricci bioRxiv 2020.10.16.341222; doi: 10.1101/2020.10.16.341222; the disclosures of which are hereby incorporated by reference in their entireties.) RNA degradation in aqueous buffers can occur in much longer time scales, but this can accelerate in the presence of magnesium (Mg²⁺) or in high pH. (See e.g., Hannah K. Wayment-Steele, Do Soon Kim, Christian A. Choe, John J. Nicol, Roger Wellington-Oguri, R. Andres Parra Sperberg, Po-Ssu Huang, Eterna Participants, Rhiju Das bioRxiv 2020.08.22.262931; doi: 10.1101/2020.08.22.262931; the disclosure of which is hereby incorporated by reference in its entirety.) Common strategies to stabilize mRNAs for in vivo stability (including appending long poly adenosine stretches; >100 As) can actually destabilize RNAs in vitro by adding additional locations for possible hydrolysis. Additionally, embedded structured segments, which are expected to stabilize RNAs against in-line hydrolysis have been shown to decrease stability of mRNAs inside human cells through a process termed structure-mediated RNA decay (SRD), involving cellular factors UPF1 and G3BP1. (See e.g., Fischer, Joseph W. et al. Molecular Cell, Volume 78, Issue 1, 70-84.e6; the disclosure of which is hereby incorporated by reference in its entirety.)

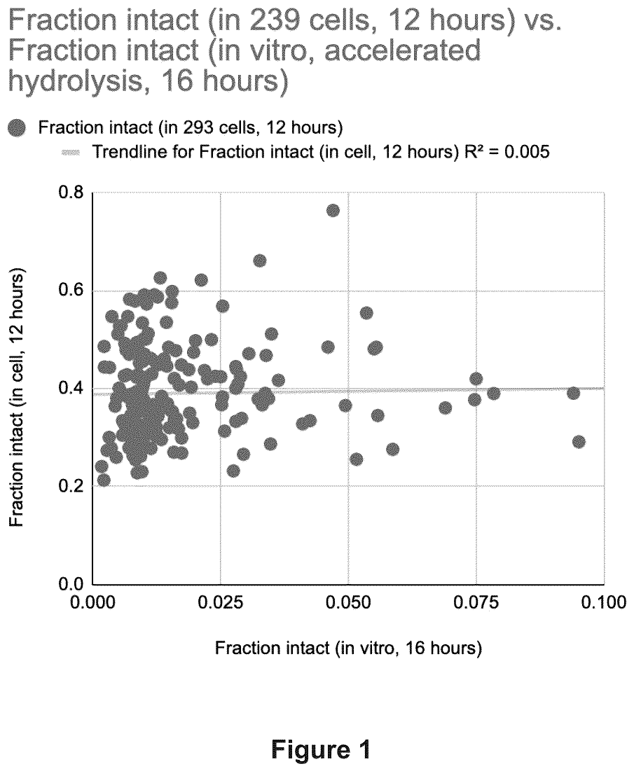

Exemplary data showing the no correlation between in vitro and in vivo stability is illustrated in FIG. 1. Specifically, FIG. 1 illustrates data from an empirical study of an mRNA library coding for nanoluciferase show that decay rates in human cells exhibit no correlation with in vitro decay rates. The in cell and in vitro stability possess an r² value of 0.0005, indicating no correlation. Such measurements were carried out using a library of 233 mRNAs of varying lengths (507-1215 nucleotides) and sequences. The measurements involve a reverse-transcription based assay to count RNAs remaining after degradation times, with strong reproducibility in ranking mRNA stabilities between time points or in replicates. In-cell measurements involved mRNAs transfected into human 293 cells. In vitro measurements were carried out under hydrolysis conditions (10 mM MgCl₂, 50 mM Na-CHES, pH 10.0, 24° C.) that accelerate hydrolysis by ~100× compared to neutral buffers without Mg²⁺.

To be effective, nucleic acid-based therapeutics, including (but not limited to) mRNA vaccines, should be stable both in vitro and in vivo to be effective for both storage and efficacy. Thus, many embodiments describe an RNA design to allow for in vivo and/or in vitro screening as well as methods to assess in vivo and/or in vitro stability.

### RNA Molecules and Design

Turning to FIG. 2, an exemplary structure for an embodiment of an RNA molecules in accordance with various is illustrated. Certain embodiments of an RNA molecule possess a 5′ cap moiety. Some embodiments utilize a 7-methyl guanosine triphosphate as the cap moiety, but various additional cap sequences are known in the art for a 5′ cap moiety. Additional embodiments possess a cap-proximal sequence for an mRNA located at the 5′ end of the mRNA. Various cap sequences are known in the art for a 5′ cap-proximal sequence. Certain embodiments use a small triplet, such GGG as the cap-proximal sequence.

Additional embodiments of an RNA molecule possess a 5′ untranslated region (5′UTR) sequence and/or a 3′UTR sequence. Certain embodiments place the 5′UTR near the 5′ end of the RNA molecule, while the 3′UTR is located near the 3′ end of the molecule. In some embodiments, the 5′UTR is located at the 3′ end of the cap, while additional embodiments place the 5′UTR directly at the 5′ end without a cap sequence. Similarly, a 3′UTR can be placed at the 3′ end of a molecule, while additional embodiments may have a tailing sequence placed 3′ of the 3′UTR. Certain embodiments select a 5′UTR and/or a 3′UTR for a variety of factors to increase stability and/or translation based on an innate sequence, while others select a 5′UTR and/or a 3′UTR for that may pose improved translation and/or stability based on a particular coding sequence of interest. Many possible 5′UTRs and 3′UTRs are known in the art, which are used in various embodiments.

Many embodiments of an RNA molecule possess a coding sequence, or CDS, located 3′ from the 5′UTR, and 5′ of the 3′UTR. In many embodiments, the CDS begins (e.g., at its 3′ end) is with a start codon (e.g., the canonical AUG and/or any other codon shown to begin translation). In many embodiments, the a CDS terminates (e.g., at its 3′ end) with a stop codon. In various embodiments the stop codon is a canonical stop codon (e.g., UAG, UAA, UGA), while further embodiments comprise noncanonical stop codons or sequences shown to terminate translation. Certain embodiments comprise more than one stop codon in the CDS.

The coding sequence is a designed sequence of interest to encode a protein or peptide of interest. In certain embodiments, the coding sequence encodes an epitope or other antigen to induce an immune response, thus allowing creation of a vaccine. In various embodiments, the protein or peptide of interest is used as a therapeutic directly, such that the protein or peptide of interest replaces or supplements a dysfunctional protein or peptide. In some embodiments, the protein or peptide of interest corrects for dysfunction of another protein or peptide. While protein coding sequences are described in the context of this exemplary embodiment, additional embodiments possess sequences for non-coding RNAs, such as RNAs that guide genome editing and/or coat chromatin. Various embodiments possess a CDS encoding a reporter gene; for example, nanoluciferase (SEQ ID NO: 1), green fluorescence protein, or any other reporter gene of interest.

Additional embodiments of an RNA molecule include a barcode to identify particular molecules based on unique sequences. Many barcode schemes are known in the art and range from 2 to 12 or more nucleotides. In many embodiments, the barcodes are 6-9 nucleotides in length. Certain embodiments select one or more barcodes from SEQ ID NOs: 2-1267.

To read barcodes, an RNA molecule can include one or more profiling sequences that can be used by PCR primers or sequencing primers to amplify and/or sequence the barcode region. In some embodiments profiling sequences are located at the 5′ and/or 3′ end of a barcode. In many embodiments, profiling sequences flank the barcode. In various embodiments profiling sequences are selected from profiling sequence 1 (SEQ ID NO: 1268) and profiling sequence 2 (SEQ ID NO: 1269).

As noted above, some embodiments of an RNA molecule possess a tailing sequence located at the 3′ end of a molecule. In various embodiments the tailing sequence is used to add a poly-A tail or other structural sequence to an RNA molecule. In some embodiments, the tailing sequence is selected as SEQ ID NO: 1270.

Structures, such as those described above in regard to FIG. 2 allow for modular and combinatorial testing of various 5′UTRs, ORFs, and 3′UTRs.

### Methods of Assessing In Vivo RNA Stability

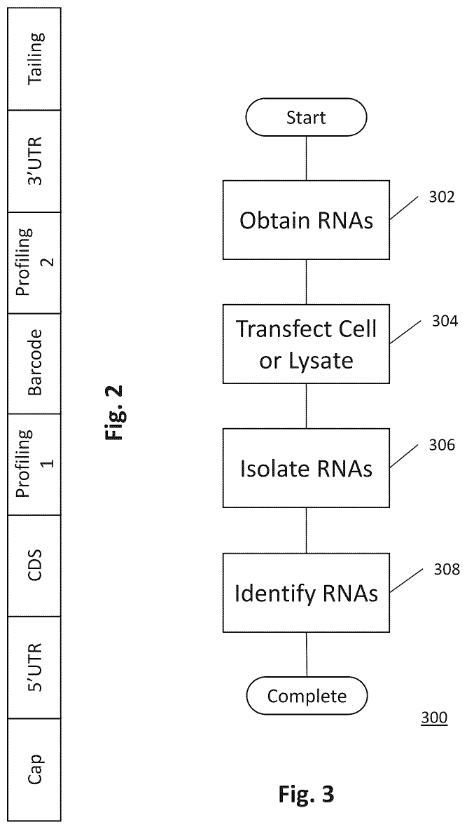

Certain embodiments assess the stability of RNA molecules, including stability within in vivo and in vitro environments. An exemplary embodiment of a method 300 to assess stability is illustrated in FIG. 3. In method 300, RNA is obtained at 302. In certain embodiments, RNA molecules are generated via in vitro transcription. Additionally, certain embodiments generate an RNA transcript and/or further modify an RNA transcript to be ready for translation (e.g., including a 5′ cap and/or a 3′ polyA tail). In various embodiments, PCR is used to amplify one or more RNA molecules, including amplification of a template library. Additional embodiments assess amplicon quality via electrophoresis, including gel (agarose and/or polyacrylamide) and/or capillary electrophoresis (e.g., ABI 3700 and/or Agilent Bioanalyzer). Further embodiments transcribe these DNA amplicons to RNA using a DNA-dependent RNA polymerase. Certain embodiments perform the in vitro transcription using commercial kits, including Thermo's T7 MEGAScript. Various embodiments modify the RNA transcripts with a 5′ cap and/or polyA tail. These modifications can be accomplished using kits, such as the Cellscript kit. Additional cleanups can be accomplished at various stages (e.g., after PCR, after transcription, and/or after modification), using columns or reagents, such as Thermo's MEGAClear columns. And, quality of the transcribed and/or modified RNAs can be accomplished via electrophoresis, including gel and capillary electrophoresis. In various embodiments, the RNA is provided as a pool of RNA sequences, where each unique RNA sequence comprises a unique barcode, such as described herein. In certain embodiments, the RNA molecules within the pool are approximately the same length.

Various embodiments transfect RNA transcripts into cells or add the transcripts to a cellular lysate at 304. In certain embodiments, transfection occurs on cultured cells or tissue, including mammalian cells, while other embodiments use yeast, bacteria, or plant cells. Some specific embodiments transfect HEK293T cells. Various embodiments incubate the transfected cells to allow for translation of the RNAs. Incubation can last between 1 hour and several days (e.g., 7-10 days) at temperatures and/or conditions to encourage cellular growth and translation. Culture media can include antibiotics or other selective reagents to prevent growth of non-transfected cells and/or contamination. Certain embodiments utilize a cellular lysate as a proxy of in vivo stress on RNA. In such embodiments, cultured cells are lysed via a known method, such as sonication, hydrodynamic stress, or any other method to generate cellular lysate. Then, the RNAs are added to the lysate and allowed to react for a period of time, such as between 1 hour and several days (e.g., 7-10 days) and at temperatures commensurate with the operating temperature for the RNA (e.g., average body temperature, 37° C.).

At 306, certain embodiments isolate RNAs based on in-cell stability. In various embodiments, RNAs are isolated from transfected cells, while some embodiments isolate the RNAs from a cellular lysate. Certain embodiments isolate RNA from transfected cells at various time points (e.g., after 1 hour, 2 hours, 3 hours, 6 hours, 12 hours, 24 hours, etc.) to create time-based fractions of RNAs. Additionally, isolated RNA molecules can be cleaned up via known procedures or kits, including isolation protocols, kits, columns, or any other know method for isolating RNA from cells or a lysate.

Various embodiments identify the RNAs based on their barcodes at 308. As noted above in relation to FIG. 2, many embodiments of RNA molecules contain a barcode sequence (e.g., SEQ ID NOs: 2-1267). The profiling sequences flanking the barcodes (e.g., SEQ ID NOs: 1268-1269) can be used to amplify the barcode or can be used as sequencing primers for barcoding reads of the RNA molecules of certain embodiments. Further embodiments utilize hybridization probes, quantitative PCR (qPCR), or any other known method with or without pooling strategies to identify which RNAs are present in timepoint based fractions.

### Determination of In Vitro RNA Stability

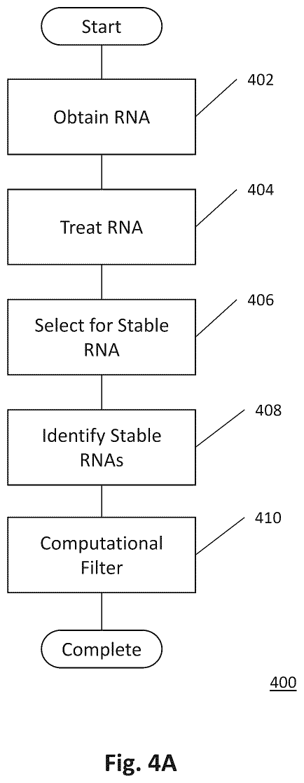

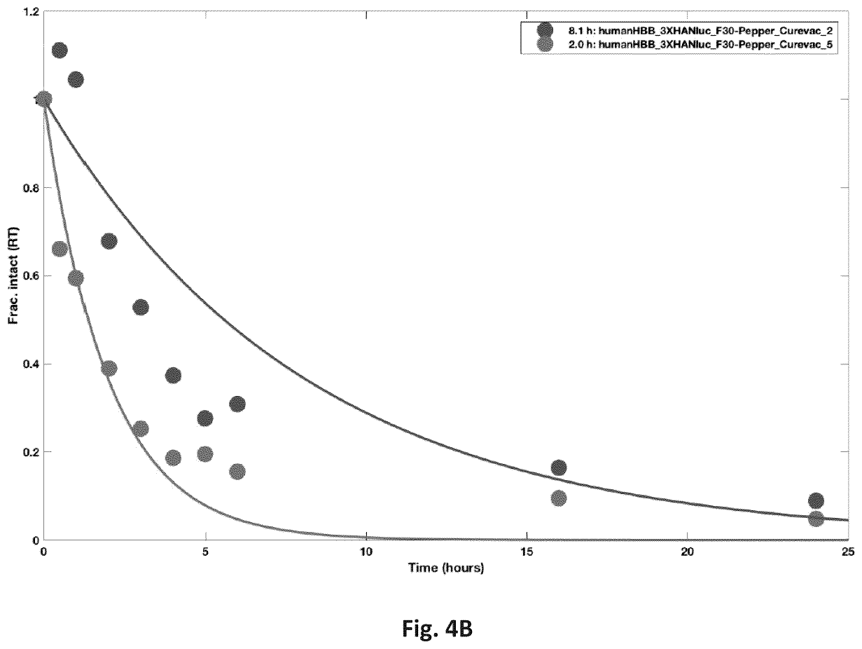

An additional challenge for RNA therapeutics, including vaccines, include the stability in storage, such as between manufacture and actual treatment or delivery to an individual. Such stability is referred to as in vitro stability, as it emphasizes stability in non-biological environments, such as in vials, syringes, or other method of storage. Various embodiments provide a method to measure in vitro stability of RNAs. Turning to FIG. 4A, a method to determine in vitro RNA stability of RNA 400 in accordance with various embodiments is illustrated. Within method 400, RNA is obtained at 402. Obtaining RNA at 402 can be accomplished via many methods, including such steps as described in regard to method 300 (FIG. 3), including the obtention of a pool of RNA molecules, where each unique RNA sequence is identifiable by a unique barcode.

At 404 of many embodiments, the RNA pool is treated or subjected to an experimental condition. The experimental conditions include any condition that may cause degradation of an RNA molecule in a storage situation, including (but not limited to) temperature, pH, presence of certain molecules and/or ions, concentration of certain molecules and/or ions, irradiation, time, buffer type, buffer concentration, and/or any other condition that can affect RNA stability. Such conditions are meant to reproduce actual conditions that can induce one or more hydrolytic events within the RNA molecules. A hydrolytic event, in accordance with various embodiments, causes a break within the RNA molecule, resulting in a broken or incomplete RNA molecule. Incomplete or broken RNA molecules may be insufficient for use as a therapeutic, thus limiting the efficacy of the molecule.

Further embodiments further select for stable RNAs in the pool at 406. In some embodiments, the selection occurs by size selecting for full length RNAs, such as through electrophoresis, including (but not limited to) agarose gel electrophoresis, polyacrylamide electrophoresis, and capillary electrophoresis. However, additional embodiments perform a nuclease digestion reaction that is selective for damaged or degraded RNA. In certain digestion reactions, the nuclease is a 5′-3′ nuclease that is inhibited by the presence of a 5′ cap moiety—XRN1 is a non-limiting example of nuclease fitting this description. Being inhibited by a 5′cap prevents any stable or undamaged RNA molecules from being digested, thus causing damaged RNA to be eliminated from the pool.

Some embodiments select for stable RNAs by performing reverse transcription PCR (RT-PCR) to amplify full length RNAs into complimentary DNA (cDNA). By creating cDNAs, downstream amplifications can utilize DNA-dependent polymerases to create sequencing libraries or other molecules for analysis. Such embodiments select for full length RNAs rather than RNAs that may have been hydrolyzed but may still be of sufficient length that electrophoresis or other methods do not remove them.

At 408, stable RNAs are identified. In various embodiments, the undigested or gel-extracted RNAs are sequenced using the barcode to identify the particular molecules that are stable. In many embodiments, cDNAs created in 406 are utilized as templates to create a sequencing library to avoid the amplification of RNAs that may be near full length.

Further embodiments integrate a computational filter 410 to remove artifacts from sequencing or other reactions that appear to show anomalous stability. For example, longer experimental conditions are generally expected to cause increased degradation. However, as illustrated in FIG. 4B, some exemplary RNA molecules show an anomalous persistence after extended times. To compensate for such artifacts, certain embodiments add a computational filter for RNA molecules in a pool. In many of such embodiments, the filter constructs a single-exponential curve for RNA molecules in a pool based on stability at various time points (e.g., 1 hour, 2 hours, 3, hours, 4 hours, 6 hours, 8 hours, etc.). For each RNA molecule, a difference between experimental fraction intact at the 24-hour time point (corresponding to >10 half-lives) and the expected intact fraction is calculated. If the residual fraction is greater than a particular threshold (e.g., 0.05), the RNA data is ignored.

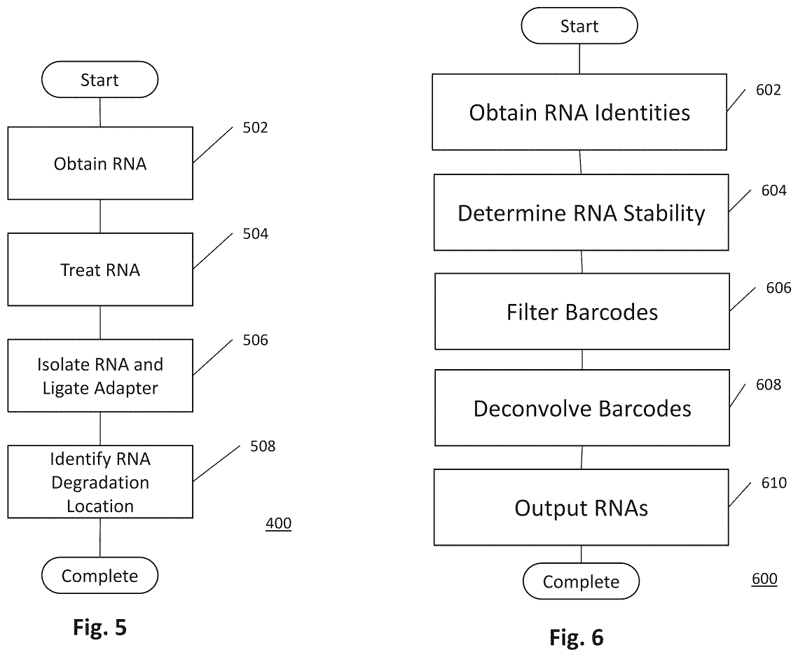

Turning to FIG. 5, additional embodiments include method 500 to identify single nucleotide resolution of RNA degradation. Such embodiments couple inline probing with sequencing to identify specific locations of hydrolysis in an RNA molecule. In method 500 of various embodiments, RNA is obtained at 502. Obtaining RNA at 502 can be accomplished via many methods, including such steps as described in regard to method 300 (FIG. 3), including the obtention of a pool of RNA molecules, where each unique RNA sequence is identifiable by a unique barcode.

In many embodiments, the RNA is treated at 504. In many embodiments, treatment is similar to 404 of method 400 or 304 of method 300. Such treatments can include variations in time, temperature, pH, buffer components, etc. Treatment in accordance with various embodiments is utilized to induce one or more hydrolytic or degradation events within one or more RNA molecules.

At 506, various embodiments isolate RNA from the treatment and ligate an adapter to the 5′ end of the molecules within the sample. The purpose of the 5′ ligation is to preserve the 3′ barcode comprised within RNA molecules of various embodiments. Various embodiments further utilize additional enzymes and reagents, such as kinases, ligases, ATP, buffers, etc. to ligate an adapter to the 5′ end of RNAs and RNA fragments after treatment at 504. In various embodiments, the adapter possesses a sequencing primer and/or provides for a polymerase amplification. By ligating the adapter to the 5′ end of a molecule, the 5′ position of the hydrolysis is preserved by the adapter for downstream analysis.

At 508, the degradation location of the RNA is identified. In many embodiments, the RNA is sequenced to identify the specific base remaining intact. Certain embodiments include building sequencing libraries or other intermediate steps to sequence RNAs, as applicable to a particular sequencing platform (e.g., Illumina, PacBio, IonTorrent, etc.).

Identifying RNAs Having Enhanced Stability and/or Translatability

Turning to FIG. 6, various embodiments identify RNA molecules possessing increased stability (in vivo and/or in vitro) in method 600. At 602, many embodiments obtain identities of RNAs present in various fractions of stability (e.g., RNAs assessed via methods 300 or 400). In various embodiments, these identities include the barcode or barcodes that identify each of the RNA molecules in a fraction and a read count of each barcode in each fraction.

At 604, various embodiments determine the stability of each RNA by identifying prevalence of each barcode in each fraction. Certain embodiments perform statistical analyses to relative prevalence of the barcode in each fraction. The presence of RNAs in fractions correlating to longer times, indicate increased stability of that particular RNA. It should be noted that barcodes with higher stability (e.g., stable for at least 7 hours) will also show stability at shorter time points (e.g., 1 hour, 2 hours, 3 hours, etc.) As such, the absence of a barcode at a particular time point (as opposed to the presence of the barcode) may be of more importance for stability analysis.

Some embodiments filter RNA molecules based on particular characteristics at 606. Particular characteristics may be specific cutoffs or minimum levels of stability or translatability of a particular barcode. For example, certain embodiments omit barcodes that have limited in vitro stability as compared to in vivo stability or vice versa.

Various embodiments deconvolve the barcodes at 608, where deconvolution involves correlating the specific RNA sequence or sequence name is produced based on the barcode sequence.

Additional embodiments output results visualizing the stability and/or translatability of particular RNA molecules. Some embodiments produce heatmaps, dot plots, or other graphs or charts to visualize in vivo and/or in vitro stability of a particular RNA.

### EXEMPLARY EMBODIMENTS

Although the following embodiments provide details on certain embodiments of the inventions, it should be understood that these are only exemplary in nature, and are not intended to limit the scope of the invention.

### Example 1: Selection of Full-Length RNA Molecules to Assess Stability

Background: The natural experimental steps to select for full-length RNAs, based on literature precedent, involve (1) ribonuclease digestion to digest degraded RNAs and leave behind intact RNAs, as happens in living cells, or (2) electrophoresis to isolate the intact RNAs. Neither of these methods work, as illustrated by the following embodiment.

Methods: An inline hydrolysis event in RNA results in two fragments. The first fragment ends in a 2′-3′ cyclic phosphate, and the other fragment begins with a 5′ hydroxyl. Initially focusing on the use of the 5′-to-3′ exonuclease Xrn1 to digest the second classes of fragments would result in elimination of any RNA that has a hydrolysis event 5′ to a barcode residing in the 3′ end of RNA molecules. Xrn1 acts 5′-to-3′ on RNAs that have a 5′ phosphate but not the initial 5′ hydroxyl left by inline hydrolysis. Thus, in preparation for an Xrn1 digestion, T4 polynucleotide kinase and ATP were used to 5′-phosphorylate degradation products.

Polyacrylamide gel electrophoresis (PAGE) and RT-PCR were also tried as an attempt to isolate or “clean up” full-length RNA molecules. RT-PCR utilized primers to capture the full-length molecules, while PAGE performed RT-PCR on only barcode regions to identify remnant RNA molecules.

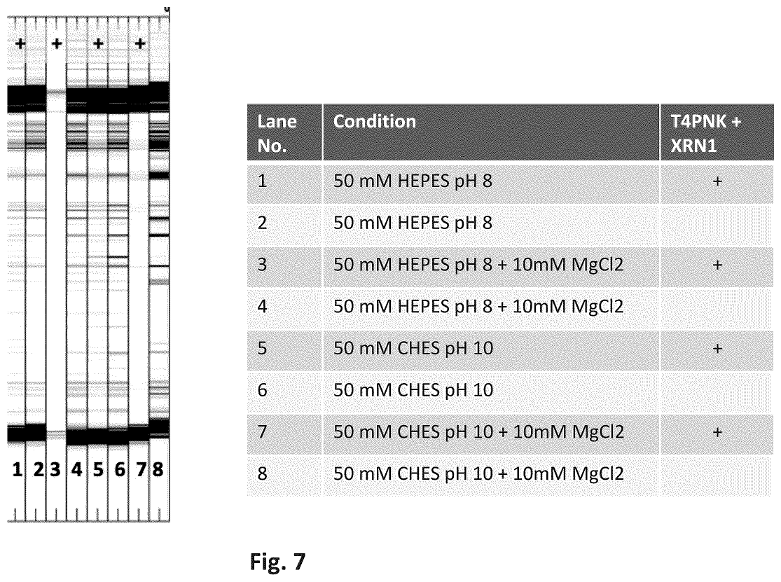

Results: FIG. 7 shows capillary electrophoresis analyzed with HiTRACE software of cDNA reverse transcribed from the P4-P6-2HP RNA that has been subject to different buffer conditions for 22 hours, including highly degrading conditions involving high pH and MgCl2. T4PNK+Xrn1 treatment ‘cleans up’ degradation products for this RNA. The bands that appear when RNA was incubated at high pH (e.g., lanes 5, 7) are hydrolyzed RNAs. The bands disappear when the RNA is then treated with T4 PNK and Xrn1, showing that the enzymatic treatment is able to ‘clean up’ the degraded RNA, and leave behind full-length RNA (the dark band at the bottom of the electropherograms).

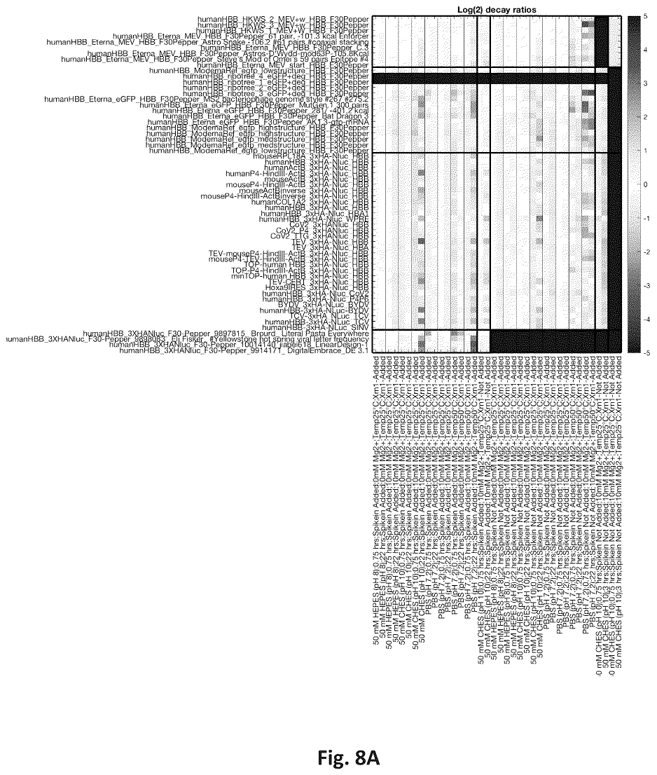

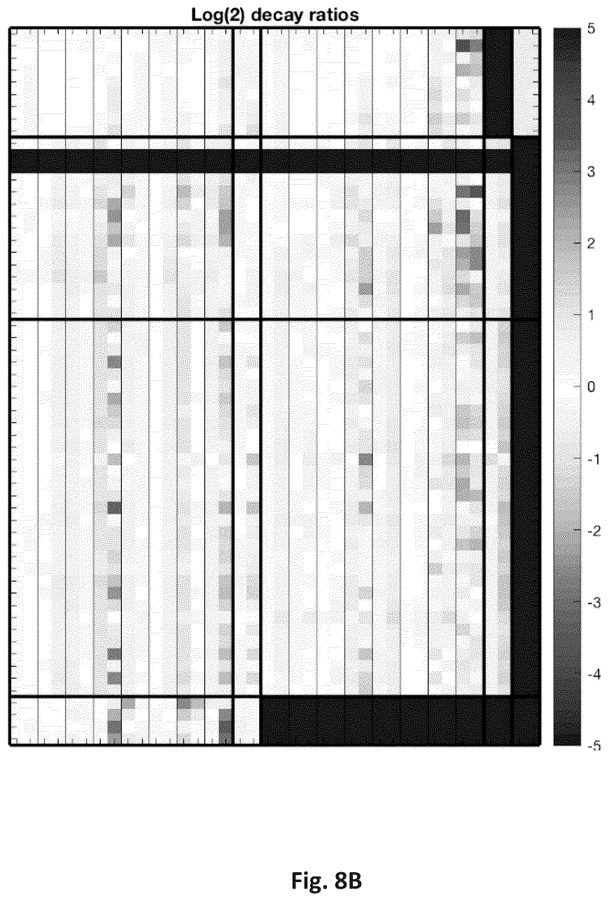

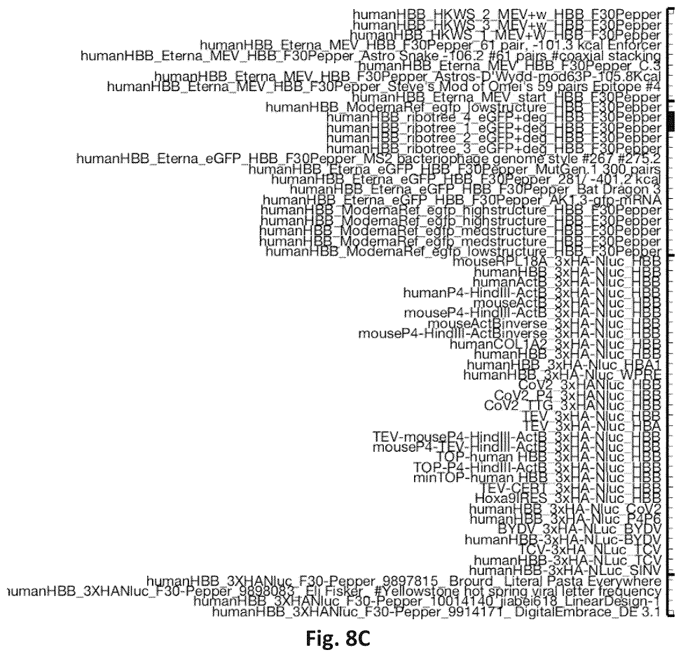

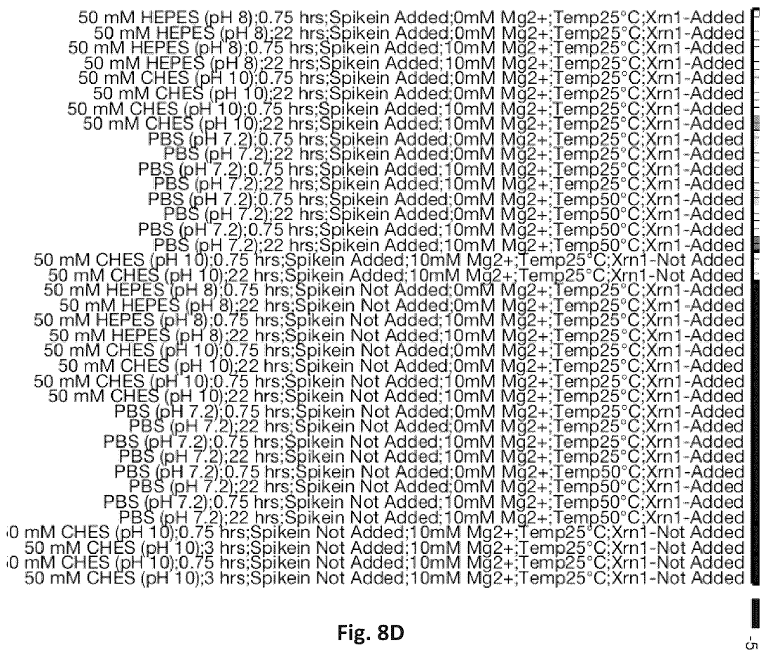

However, paradoxical results were observed when the same T4 PNK+Xrn1 was used to destroy hydrolytic degradation products of a library of >50 RNAs that had been ‘aged’ in different buffers, including a high pH (CHES, pH 10.0) condition expected to produce severe degradation. A single RT-PCR was used to select just the ‘barcode’ region of the RNAs. Counts of those RNAs were compared to a spike-in control that was not degraded; normalization of these numbers to samples that were not subjected to degradation. FIGS. 8A-8D illustrate a heatmap showing RNAs dropping out after degradation for 22 hours, but only by 2-4 fold. Specifically, FIG. 8A illustrates a full view of the heatmap and column and row labels; while FIG. 8B illustrates an enlarged view of the heatmap; FIG. 8C illustrates the row labels; and FIG. 8D illustrates the column labels. It was expected that >99% of the RNAs would be gone by that point based on one-by-one characterization of these RNAs. In addition, IT expected that RNAs with different 3′ UTRs would give similar degradation rates, but instead it was observed that variation of the apparent degradation rate depending on 3′ UTR identity (compare strength of blue pixels in TEV_3xHA-NLuc-HBA to TEV_3xHA-NLuc-HBB row in FIGS. 8A-8D).

The observations in FIGS. 8A-8D suggest that Xrn1 digestion might be incomplete depending on the RNA sequence; it is indeed known that some RNA sequences and structures block Xrn1. If any degraded RNA can survive the Xrn1 treatment, it can get amplified by RT-PCR.

Conclusions: Certain RNA molecules show anomalous degradation patterns after cleanup. However, such anomalies are removed by an additional RT-PCR step that selects for full-length molecules, rather than relying on degradation to filter out hydrolysis products.

### Example 2: Computation Filter for Artifacts

Background: Certain RNAs still showed full-length appearance after sequencing. Such RNAs could be due to mis-priming during RT-PCR (e.g., Example 1). Many of the artifacts occurred in the GC-rich RNAs, which are known to be prone to mispriming in PCR reactions. Indeed, for some of these RNAs, use of different primer pairs resulted in data where the fraction intact did drop to 0 at long timepoints, supporting the hypothesis that these RNAs were susceptible to RT-PCR artifacts that depend on primer pairs. In addition, some of these anomalous RNAs were characterized using one-by-one synthesis, degradation, and capillary electrophoresis, and discovered that they were completely degraded by 10 hours, as expected, with no detectable fraction intact after that timepoint.

Methods: To generate a computation filter, data for each RNA was fit to single-exponential curves, then for each RNA, the difference between the experimental fraction intact at 24 hours (corresponding to >10 half-lives) was calculated, and the fraction intact expected at that time point based on the single-exponential fit. If this ‘residual’ was greater than 0.05, the RNA data was not considered for further analysis.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1-16. (canceled)

17. A method to determine RNA stability, comprising:
obtaining a pool of RNA molecules, wherein each RNA molecule is uniquely encoded with a barcoding sequence and each barcoding sequence is flanked by at least one profiling sequence;
treating the pool of RNA molecules under an experimental condition; and
isolating the pool of RNA molecules at a specified timepoint to generate a fraction of RNA molecules showing stability under the experimental condition for the specified timepoint.

18. The method of claim 17, further comprising sequencing the barcode sequence of each RNA molecule in the fraction to identify the presence of each RNA molecule in the fraction of RNA molecules.

19. The method of claim 18, further comprising determining stability of the RNA molecules associated with each barcode sequence in the fraction by identifying the prevalence of each barcode in the fraction.

20. The method of claim 17, wherein the treating step comprises transfecting the pool of RNA molecules into a collection of cells.

21. The method of claim 20, wherein the cells are selected from mammalian cells, yeast cells, bacteria cells, and plant cells.

22. The method of claim 17, wherein the treating step comprises adding the pool of RNA molecules to a cell lysate.

23. The method of claim 17, wherein the treatment condition is selected from temperature, pH, presence of certain molecules, presence of certain ions, concentration of certain molecules, concentration of certain ions, irradiation, buffer type, and buffer concentration.

24. The method of claim 17, further comprising size selecting for full-length RNA molecules.

25. The method of claim 24, wherein size selecting comprises one or more of agarose gel electrophoresis, polyacrylamide gel electrophoresis, and capillary electrophoresis.

26. The method of claim 24, wherein size selecting comprises treating the RNA molecules with a 5′-3′ nuclease that is inhibited by the presence of a 5′ cap moiety.

27. The method of claim 24, wherein size selecting comprises performing reverse transcription PCR to amplify full-length RNA molecules.

28. The method of claim 17, wherein the isolating step further comprises isolating the pool of RNA molecules at a second specified timepoint to generate a second fraction of RNA molecules showing stability under the experimental condition for the specified timepoint.

29. A method to identify a degradation site within an RNA molecule, comprising:
obtaining a pool of RNA molecules, wherein the RNA molecules encode for a sequence of interest,
treating the pool of RNA molecules under an experimental condition to degrade the RNA molecules in the pool of RNA molecules; isolating the pool of RNA molecules at a specified timepoint;
ligating an adapter to one end of the degraded RNA molecules in the pool of RNA molecules; and
sequencing the ligated and degraded RNA molecules in the pool of RNA molecules to identify the degradation locations in the pool of RNA molecules.

30. The method of claim 29, wherein the adapter is ligated to the 5′ end of the degraded RNA molecules.

31. The method of claim 29, wherein the treatment condition is selected from temperature, pH, presence of certain molecules, presence of certain ions, concentration of certain molecules, concentration of certain ions, irradiation, buffer type, and buffer concentration.

32. The method of claim 29, wherein the pool of RNA molecules comprises a plurality of sequences of interest, wherein each sequence of interest is uniquely encoded with a barcoding sequence and each barcoding sequence is flanked by at least one profiling sequence.

33. A method to determine in vitro RNA stability, comprising:
obtaining a pool of RNA molecules, wherein each RNA molecule is uniquely encoded with a barcoding sequence and each barcoding sequence is flanked by at least one profiling sequence;
subjecting the pool of RNA molecules to an in vitro experimental condition configured to cause hydrolytic degradation of the pool of RNA molecules;
isolating a fraction of the pool of RNA molecules at a specified timepoint;
performing a selection on the isolated fraction to select for full length RNA molecules;
sequencing the barcoding sequence of each RNA molecule of the selected full length RNA molecules to identify RNA molecules in the isolated fraction at the specified timepoint; and
determining, based on a prevalence of the identified RNA molecules in the isolated fraction at the specified timepoint, in vitro RNA stability of each RNA molecule in the pool of RNA molecules at the specified timepoint.
